(19) United States
(12) Patent Application Publication
(10) Pub. No.: US 2017/0105832 A1
(43) Pub. Date: Apr. 20, 2017

(54) POROUS MEMBRANE STRUCTURES AND RELATED TECHNIQUES

(71) Applicant: Kenneth Samuel Rosenblum, Minneapolis, MN (US)

(72) Inventor: Kenneth Samuel Rosenblum, Minneapolis, MN (US)

(21) Appl. No.: 15/295,621

(22) Filed: Oct. 17, 2016

Related U.S. Application Data
(60) Provisional application No. 62/243,503, filed on Oct. 19, 2015.
(60) Provisional application No. 62/242,225, filed on Oct. 15, 2015.

(51) Int. Cl. A61F 2/02; C25B 11/04; C12N 5/071; A61M 37/00; A61N 1/20; A61N 1/05; C25B 1/10; A61M 31/00

(52) U.S. Cl. CPC A61F 2/022; C25B 1/10; C25B 11/0415; A61M 31/002; A61M 37/00; A61N 1/205; A61N 1/05; C12N 5/0676; A61M 2202/09; A61M 2205/0272; C12N 2535/00; C12N 2539/00

(57) ABSTRACT

A conductive porous fabric can be formed, such as by using a template material. The porous fabric can be conductive, such as thick enough to be self-supporting, or supported such as by another structure. The porous fabric can be used in implantable or percutaneous applications, such as to provide an immunoisolation barrier. In another example, the fabric can be coupled to an electric potential, such as to facilitate gas evolution when the porous fabric is located in an aqueous medium. Such gas evolution can be used for various purposes, such as to maintain living cell viability by providing oxygen, or for self-cleaning. Illustrative examples of porous fabric materials include gold, platinum, palladium, iridium, niobium, or a form of carbon such as graphene.

### PRIORITY APPLICATIONS

This application claims the benefit of priority to U.S. Provisional Application Ser. No. 62/243,503, filed Oct. 19, 2015, and to U.S. Provisional Application Ser. No. 62/242,225, filed Oct. 15, 2015, the contents of both which are incorporated hereby by reference in their entireties.

### BACKGROUND

Structures including porous membranes are believed useful in a broad range of applications including medical devices or engineering-related fields such as filtration or water treatment. Porous membranes can provide surfaces or barriers established to control diffusion or permeation by other species on the basis of geometric or chemical characteristics.

As an illustrative example, over the past three decades or so, attempts have been made to cure Type I diabetes by performing allograft or xenograft islet cell transplants. While there is a moderate rate of success when the patient is immunosuppressed for other reasons (such as kidney transplant), such islet transplantation has shown little success where immunosuppression has not been used. Therefore, to provide immunoisolation, one approach can include placing islets behind various types of immunoisolation barriers to prevent host rejection.

For islet transplantation, various isolation barriers have been explored including porous materials (e.g., nanoporous materials) such as anodic aluminum oxide (alumina), titanium oxide (titania) nanotubes, porous silicon, nanostructured ceramics and track-etched membranes. In one approach, a relatively thick hydrogel polymer such as an alginate can be used. Hydrogels such as alginate) have many remarkable biocompatible properties, but a porous hydrogel membrane is generally about 50 micrometers to about 100 micrometers thick in order to provide structural stability. A typical diffusion distance from a blood capillary to an islet is about two to about three micrometers. Accordingly, long-term transplanted islet survival relying on porous hydrogel membranes has been difficult to achieve. But a hydrogel approach can have other drawbacks, such as feedback disrupted by a long diffusion distance established by the thick hydrogel layer, unwanted broad pore size distribution, or poor chemical resistance. Moreover, transplanted islets encapsulated in materials as mentioned above are generally placed in anatomic areas where materials are exchanged between islet cells and interstitial fluid, not arterial blood. Accordingly, islet survival is less likely because O₂ partial pressures in interstitial fluids are much lower than in arterial blood. Furthermore, glucose-insulin system feedback issues arise due to the relatively slow transit time from the interstitial tissue to the systemic bloodstream.

In sum, despite extensive efforts, long-term insulin free status for an islet transplate recipient has still not been attained.

### SUMMARY

### DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document,

FIG. 1 illustrates a view of porous fabric in accordance with at least one example of the present disclosure;

FIG. 2 illustrates an enlarged view of the porous fabric of FIG. 1 in accordance with at least one example of the present structure;

FIG. 3A illustrates a cross-sectional view of a self-supporting porous fabric in accordance with at least one example of the present disclosure;

FIG. 3B illustrates a view of a reinforced porous fabric in accordance with at least one example of the present disclosure;

FIG. 3C illustrates a view of a reinforced porous fabric in accordance with at least one example of the present disclosure;

FIG. 4 illustrates a top view of a porous foil in accordance with at least one example of the present disclosure;

FIG. 5 illustrates a top view of a porous foil in accordance with at least one example of the present disclosure;

FIG. 6 illustrates a top view of a porous foil in accordance with at least one example of the present disclosure;

FIG. 7 illustrates a side view of a porous foil in accordance with at least one example of the present disclosure;

FIG. 8 illustrates one example of an application for a porous fabric membrane in accordance with at least one example of the present disclosure;

FIG. 9 illustrates another example of an application for a porous fabric membrane in accordance with at least one example of the present disclosure;

FIG. 10 illustrates another example of an application for a porous fabric membrane tray in accordance with at least one example of the present disclosure;

FIGS. 11A and 11B each illustrate another example of an application for a porous fabric membrane tray in accordance with at least one example of the present disclosure;

FIG. 12 illustrates another example of an application for a porous fabric membrane having a spiral porous fabric separator in accordance with at least one example of the present disclosure;

FIGS. 13A and 13B illustrate other example of an application for a porous fabric membrane incorporated into an implant in accordance with at least one example of the present disclosure;

FIG. 14 illustrates another example of an application for a porous fabric membrane including anti-fouling or self-cleaning capabilities in accordance with at least one example of the present disclosure; and

FIGS. 15A and 15B illustrate other examples of an application for an electrolytic device in accordance with other examples of the present disclosure.

### DETAILED DESCRIPTION

Porous substrates and coatings have been proposed for use as immunoisolation barriers, and porous substrates having a large surface area have also been considered for a variety of other applications including nano-manufacturing (e.g., nanofabrication), energy harvesting, materials and structures for use in integrated electronic or electro-optical circuits, biological or chemical sensing, orthopedic implants, and controlled drug delivery. Generally, porous surfaces can be fabricated with varying degrees of one or more of pore size, pore distribution or lattice configuration, and pore density. A porous surface can be manipulated or modified, such as to provide desired chemical properties (e.g., hydrophobicity or reactivity). For example, a porous structure can be one or more of chemically functionalized or clad to suit various applications.

The present inventor has recognized that various existing approaches for forming immunoisolation barriers have failed for a variety of reasons, such as including:
(1) Transplanted cells (e.g., islets) dying due to lack of oxygen and other nu rents;
(2) Transplanted cells dying from host rejection if antibodies are not blocked or suppressed;
(3) Large diffusion distances in various proposed immunoisolation materials causing glucose/insulin system feedback errors in applications involving islets; and
(4) biofouling, thrombosis and immunoactivation of the membrane.

Described herein are various structures and processes that can include use or fabrication of “fabric” materials, such as conductive porous fabric materials. The structures described herein can include a nanoporous fabric membrane that is self-supporting, or can include a combination of layers including a nanoporous fabric membrane layer. Also described herein are techniques that can include electrolysis to evolve a gas in an aqueous medium. The drawings, which are not necessarily drawn to scale, illustrate generally by way of example, but not by way of limitation, various embodiments discussed in the present document.

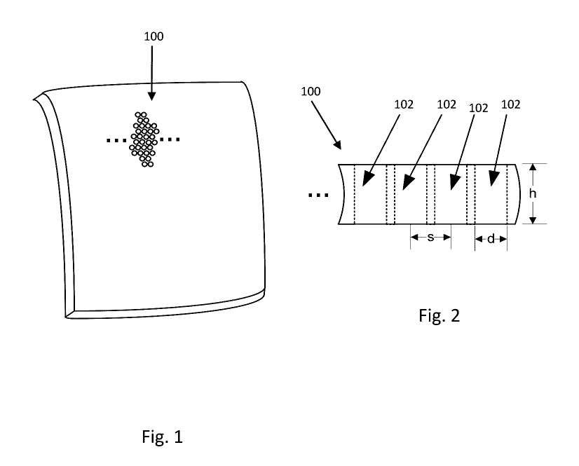

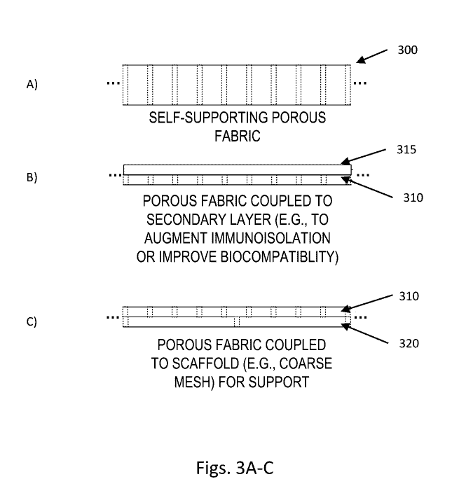

As illustrated in at least FIGS. 1-2, porous materials 100 described herein can include pores 102 having a pore size (e.g., d) that is tunable to between about 3 nanometers (nm) and about 1 micrometer. A porous fabric having pore size in the range of nanometers or tens of nanometers can be referred to generally as “nanoporous.” With additional reference to FIG. 3A, a porous fabric can be self-supporting, such as at a thickness (e.g., h) of about 1 micrometer, or even a lesser thickness, such as between about 100 nm and about 500 nm. Such a self-supporting thickness is about 1 to 2 orders of magnitude lesser in thickness than a corresponding self-supporting thickness of a hydrogel-based membrane, due at least in part to a high aspect ratio of the membrane (e.g., where a thickness of the material is much greater than a diameter of the pores). With reference to FIGS. 3B-3C, the porous fabric layer 310 can be thinner when reinforced or supported by another structure such as, for example and without limitation, a secondary layer 315 or a scaffold 320. For example, the porous fabric layer can include a thickness of about 30 nm to about 50 nm when reinforced or otherwise supported. It is contemplated either a secondary layer 315 or a scaffold 320 can, for example and without limitation, serve to augment immunoisolation, improve biocompatibility, provide support, and the like.

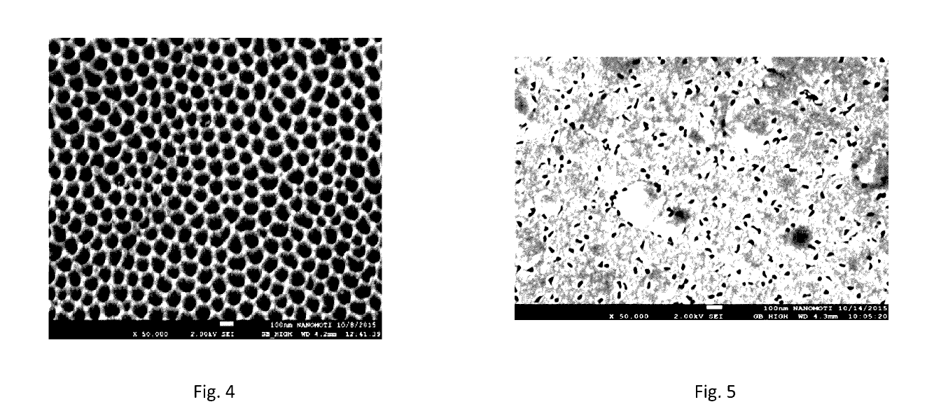

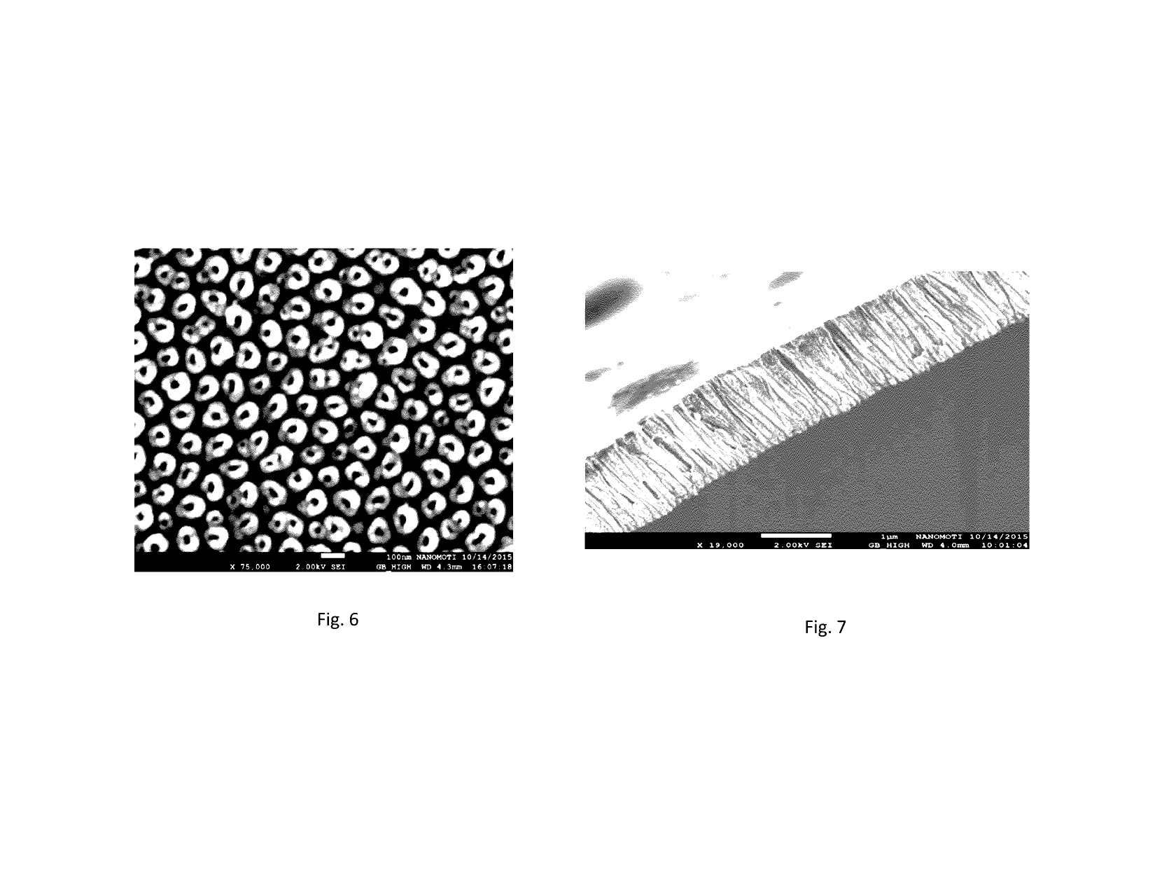

As an illustrative example, the porous fabric membrane can include various materials, such as a conductive material. For example, one or more of gold or platinum can be used, such as to provide biocompatibility, including anti-thrombotic and anti-immunogenic characteristics. Techniques for forming a porous fabric membrane can include one or more of the examples included in the accompanying Appendix, the examples depicted in the micrographs of FIGS. 4-7, or one or more of the following references, each of which are hereby incorporated herein in their respective entireties:
1. Hideki Masuda et al. 1991 Fabrication of Porous TiO2 Films Using Two-Step Replication of Microstructure of Anodic Alumina, Jpn. J. Appl. Phys. 31 1775. DOI:10.1143/JJAP.31.L1775
2. Hideki Masuda et al. 1995. Ordered Metal Nanohole Arrays Made by a Two-Step Replication of Honeycomb Structures of Anodic Alumina. Science 9 Jun. 1995: Vol. 268 no. 5216 pp. 1466-1468. DOI: 10.1126/science.268.5216.1466
3. Hideki Masuda et al. 1993. Preparation of Microporous Gold Films by Two-Step Replicating Process Using Anodic Alumina as Template. Bull. Chem. Soc. Jpn.: Vol. 66 No. 1 pp. 305-311. DOI: 10.1246/bcsj.66.305

Without being bound by theory, use of techniques mentioned and described herein can provide porous fabric material having a pore configuration including good uniformity of size and shape, and it is believed that the fabrication process for producing such a porous fabric can be scaled inexpensively. FIGS. 4-7 illustrate some examples of gold “nano”-porous foils fabricated similarly to the techniques mentioned herein are shown below, as imaged using scanning electron microscopy (scale shown by units (e.g., “100 nm” and white bar).

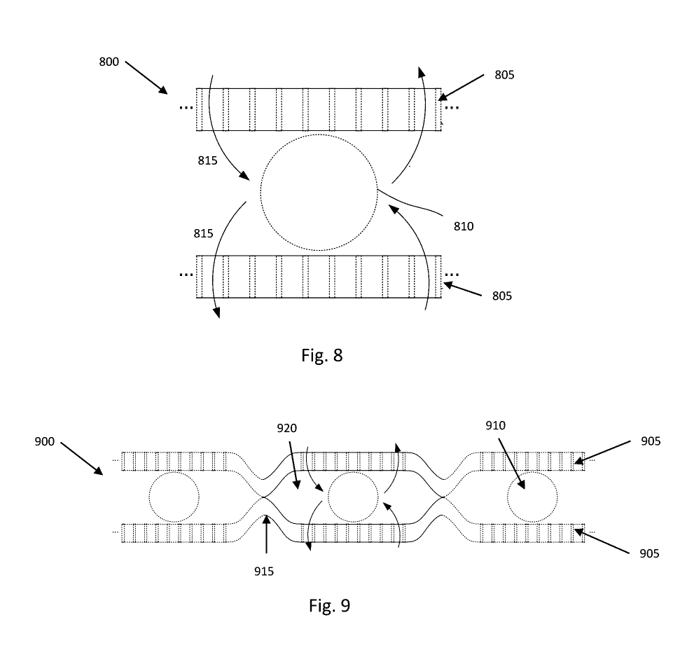

One illustrative example of an application for a porous fabric membrane shown in FIG. 8 can include use as an immunoisolation layer, such as to facilitate transplant of islet cells without requiring immunosuppression. For example, various structures 800 such as a subcutaneous or intravascular implant or fistula can include a porous fabric membrane 805. The porous fabric membrane 805 can provide an immunoisolation barrier precluding antibody and other immunologic attack of transplanted sequestered islet cells 810, but can allow diffusion or permeation by blood, interstitial, or other bodily fluid 815 to facilitate natural feedback, such as eliciting insulin secretion by the islet cells in a manner mimicking normal pancreatic function. To further supplement the biocompatibility or anti-immunogenic properties of the porous fabric 805, a hydrogel layer or other selective barrier can be coupled to the porous fabric. In this manner, a beneficial immunoisolation property of the secondary selective barrier can be provided, but avoiding the suppression of oxygen or nutrient permeation as would generally be associated with a thicker structure lacking the porous fabric. In yet another example, the porous fabric need not be self-supporting, and can be coupled (e.g., clad) to a support structure such as a coarse mesh (e.g., stainless steel).

As further illustrated in FIG. 9, various approaches can be used to enclose regions of the porous fabric 905 of the structure 900 containing one or more islet cells 910, such as can include crimping 915 or shaping the porous fabric or a stack of layers including the porous fabric to define islet cell regions 920:

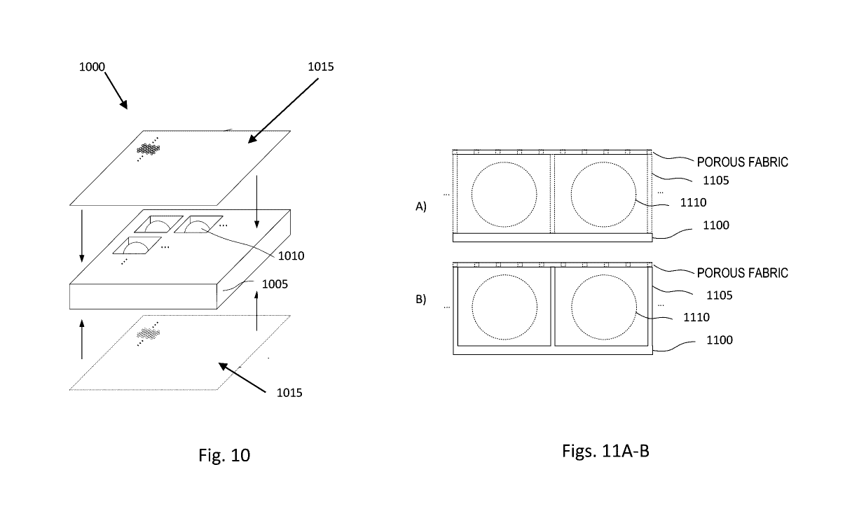

In another approach illustrated in FIG. 10, a structure 1000 comprising a tray or mesh 1005 can be formed, such as using a polymer material (e.g., silicone or polypropylene), or another material. The tray or mesh 1005 can define islet regions 1010 containing one or more islets each, and a porous fabric 1015 (or a stack of materials including the porous fabric) can be coupled to the mesh or tray to provide diffusion “windows” for interaction between blood and cells located within each defined holding region an open tray or mesh 1005 is used, another layer comprising a porous fabric 1015 can be coupled to a face of the tray or mesh opposite the first porous fabric layer.

As illustrated in FIGS. 11A-B, the mesh or tray need not be one material. For example, abuse or substrate material 1100 such as a biocompatible metal film can be formed, and the partitions 1105 defining the islet cell holding regions 1110 can be coupled to the substrate. In another example, the partitions 1105 can be defined such as using deposition or lithographic techniques, such as using the same material as the base or substrate 1100.

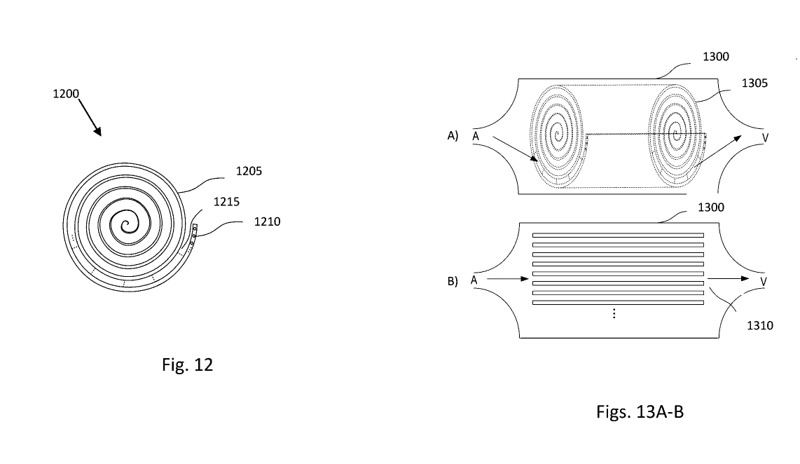

Each of the examples above can be used to provide a planar immunoisolation structure. Such planar structures can be enclosed in an implantable housing, such as an implantable housing comprising an array of such planar structures. In yet another example illustrated in FIG. 12, a housing 1200 comprising a spiraled porous fabric 1205 defining a plurality of islet regions 1210, each islet region separated by at least one separator 1215 and containing immunoisolated cells can be provided, such as to provide a large surface area within a small amount of space. The housing 1200 can be configured for implant subcutaneously or in an abdominal cavity, for example, such as for use in transplant of islet cells to provide a structure mimicking normal pancreatic function In another example illustrated in FIGS. 13A-13B, an implant 1300 comprising fistula or AV shunt can be formed, such as to enhance performance the implant by providing access to vascular blood flow: In one example, the implant 1300 can comprise a roll 1305 including immunosequestered islets and porous fabric such as that depicted in FIG. 12. In another example, the implant 1300 can include an array of immunosequestered islets and porous fabric stacks 1310.

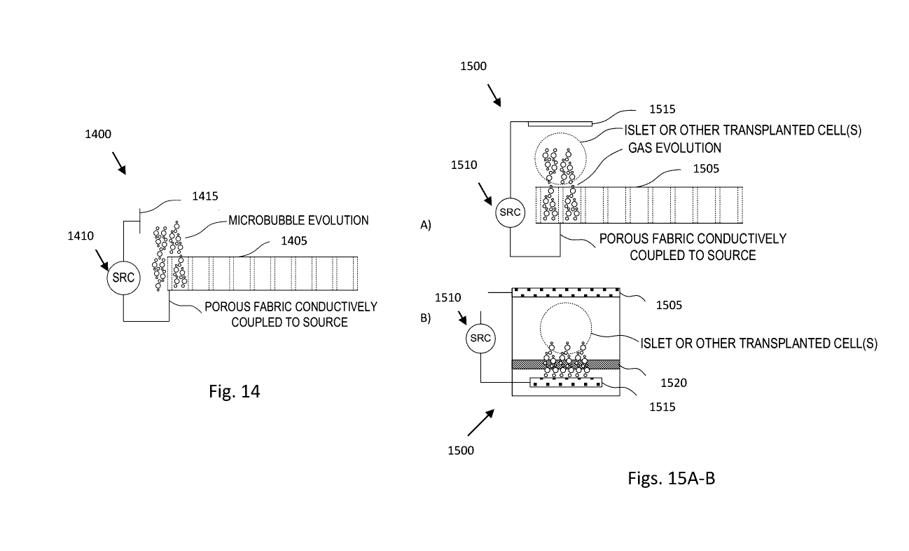

In another example illustrated in at least FIG. 14, a device 1400 comprising porous fabric 1405 can include anti-fouling or self-cleaning capabilities. For example, challenges exist in the field of implantable sensors such as due to one or more of protein adsorption or bio-fouling of the sensor surface. Protein adsorption can function as a pathway initiator for other processes that tend to foul membranes, such as facilitating thrombosis. A porous fabric 1405, such as comprising a conductive material as described herein, can be configured to achieve self-cleaning. For example, metals such as gold, platinum, or palladium can be conductively coupled to an electric potential 1410 when surrounded by an aqueous medium. A counter electrode 1415 located elsewhere can be placed in the medium. Depending on the polarity of the potential applied to the conductive porous fabric, the surrounding medium will dissociate into hydrogen or oxygen gas, forming microbubbles that nucleate on the porous fabric. Such microbubbles can be extremely effective in cleaning the surface of the porous fabric of debris. In an implantable or percutaneous application, if such microbubbles are transported away via venous blood flow to be transported to the lungs), or are left to be reabsorbed in interstitial tissue, such microbubble evolution is believed harmless.

In yet another illustrative example, a conductive material can be coupled to a source as mentioned above, for electrolysis to evolve oxygen for other purposes. For example, evolved oxygen can be supplied or otherwise directed to living cells or tissue. As an illustration, a diffusion distance between an oxygenated medium such as blood and transplanted cells may stress or even kill transplanted cells over time. In one approach, oxygen replenishment can be accomplished such as by supplying oxygen from elsewhere, such as using a hypodermic needle or other access pathway to introduce oxygen to immunoisolated cells. But, such an approach can present various challenges, such as inconvenience, risk of cell death if an oxygen replenishment is not performed in a timely manner, or risk of infection if a percutaneous pathway is used to introduce the oxygen. By contrast, the present inventor has recognized, among other things, that oxygen can be evolved locally (e.g., in-vivo) using electrolysis, such as to evolve oxygen within or nearby interstitial tissue, subcutaneous tissue, the peritoneal cavity, or another location such as within the vasculature or within a fistula.

As illustrated in at least FIGS. 15A-B, a device 1500 configured to generate oxygen using electrolysis is provided. In one example, oxygen generation is accomplished using an assembly comprising a conductive surface 1505 comprising an inert metal surface including one or more of gold, platinum, palladium, stainless steel, iridium or niobium, as illustrative examples. Other conductive materials can be used, such as can include one or more forms of carbon, including graphite, graphene, or diamond. The surface of the conductor 1505 can be modified or structured to facilitate nucleation of oxygen bubbles at specified locations or having other specified characteristics. The conductive surface 1505 need not be porous or a fabric, where the surface is being used for oxygen generation rather than as a membrane. The source (SRC) 1510 used to drive the electrolysis can include a DC source or another source having a periodic or other waveform. The surface where oxygen is generated (e.g., a first electrode) can be located nearby living cells such as within a cell holding region or separated from a holding region such as by a semi-permeable membrane 1520 (as shown illustratively in the examples below). A second electrode 1515 can be located elsewhere, such as within an aqueous medium in proximity to the first electrode 1505.

In an example, a porous membrane can be used to provide an immunoisolation barrier between implanted cells and surrounding tissue or blood, and the immunoisolation barrier can be arranged as a first electrolysis electrode. A second electrode can be located elsewhere. Either the first or the second electrode can be assigned a polarity to achieve oxygen evolution, and such a polarity or applied voltage magnitude can be varied.

The illustrative examples mentioned above refer to evolution of oxygen locally nearby living cells in-vivo, such as to maintain cell viability. Other approaches can also be used, such as use of electrolysis in a solution to evolve another gaseous species (e.g., hydrogen). As yet another example, an electrolysis cell can be located elsewhere, such as to provide in-vitro gas formation, which can then be supplied to another location. Electrolytic gas formation using porous membranes can be useful for other applications, such as to facilitate wound healing, treat a disease, or to generally affect a normal or abnormal bodily function. Also, while various examples above refer to formation of gas bubbles, the techniques mentioned above can also be used to adjust a concentration of dissolved gas in an aqueous medium, such as by modulating a rate at which gas is evolved or by controlling other characteristics such as bubble geometry or density. In each of the examples above, gas can be generated intermittently or continuously, such as using a DC source, or using a time-varying source such as including current polarity reversal.

### Various Notes & Examples

Each of the non-limiting examples described herein can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at leas n part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A structure including a conductive porous fabric layer as shown and described herein.

2. The structure of claim 1, wherein the conductive porous fabric layer is coupled to a mesh or tray, the mesh or tray defining immunoisolated regions for cells.

3. The structure of claim 2, wherein the immunoisolated regions are sized and shaped to hold pancreatic islet cellular structures.

4. A structure including a conductive porous fabric layer as shown and described herein, wherein the conductive porous fabric layer is coupled to a first polarity of an electric potential.

5. The structure of claim 4, wherein the conductive porous fabric layer is surrounded by an aqueous medium and a polarity of the electric potential opposite the first polarity is coupled to the aqueous medium.

6. The structure of claim 5, wherein the electric potential is generated by a power source, the power source establishing a potential between the surface of the conductive porous fabric layer and the surrounding aqueous medium sufficient to cause evolution of microbubbles on the surface of the conductive porous fabric layer.

7. The structure of claim 6, wherein the potential is time-varying.

8. The structure of claim 7, wherein the time-varying potential is established to control one or more of a rate or type of gaseous species evolved on the surface of the conductive porous fabric layer.

9. A structure including a conductive first electrolysis electrode;
a second electrolysis electrode; and
a source coupled to the first and second electrolysis electrodes.

10. The structure of claim 9, wherein the first electrolysis electrode is configured to provide oxygen to one or more living cells.

11. The structure of claim 10, wherein the first electrolysis electrode is fluidically coupled to a location including the one or more living cells.

12. The structure of claim 9, wherein the first electrolysis electrode is configured to provide oxygen to one or more of nearby interstitial tissue, nearby subcutaneous tissue, a peritoneal cavity, a location within the vasculature, or a location within a fistula.

13. The structure of claim 9, wherein the first electrolysis electrode comprises one or more of gold, platinum, palladium, stainless steel, iridium or niobium.

14. The structure of claim 9, wherein the first electrolysis electrode includes a modified surface to control one or more as evolution characteristics.
